SEQUENCE LISTING (3 sequences)

SEQ ID NO: 1 - DNA, 25 bp, Artificial Sequence
  Description of Artificial Sequence internal transposon specific primer
gggaattcttgaacgagttgtcctc

SEQ ID NO: 2 - DNA, 24 bp, Artificial Sequence
  Description of Artificial Sequenceinternal transposon specific primer
gggaattcagtgtaaattttgcgc

SEQ ID NO: 3 - DNA, 20 bp, Artificial Sequence
  Description of Artificial Sequenceprimer based on the terminal repeat of the transposon
gggaattcgctccgcccctg

(19) United States
(12) Patent Application Publication
(10) Pub. No.: US 2005/0059057 A1
(43) Pub. Date: Mar. 17, 2005

(54) Method of parallel screening for insertion mutants and a kit to perform this method

(76) Inventors: Tamara Maes, Barcelona (ES); Tom Gerats, Gent (BE)

Correspondence Address: TRASK BRITT, P.O. BOX 2550, SALT LAKE CITY, UT, 84110, US

(21) Appl. No.: 10/901,645

(22) Filed: Jul. 28, 2004

Related U.S. Application Data
(63) Continuation of application No. 09/578,361, filed on May 24, 2000, now abandoned.
Application No. 09/578,361 is a continuation of application No. PCT/EP98/07551, filed on Nov. 23, 1998.

(30) Foreign Application Priority Data
Nov. 25, 1997 (EP) 97203680.0

(51) Int. Cl. C12Q 1/68

(52) U.S. Cl. 435/6

(57) ABSTRACT

The current invention is a novel approach termed “parallel screening” which allows simultaneously screening of a population for insertions in all genes cloned from that or a closely related organism. In order to test this approach, the flowering plant Petunia hybrida was used as a model system. Petunia hybrida line W137 contains a high copy number of the endogenous transposable element dTph1 and has been previously presented as a genetic tool. A 3D library of the plant genomic DNA of 1000 Petunia hybrida W137 plants was generated. The 3D library consists of 30 pools of DNA from 100 plants each. These were used to generate 30 pools of insertion flanking sequences by nested iPCR using a set of transposon-specific primers or by Transposon Display PCR. Insertions into a gene were detected by hybridizing the amplified insertion flanking sequences fixed to a filter with a gene-specific probe, an approach termed simple screening for insertion elements. Alternatively, the amplified insertion element flanking sequences were labeled and used as a probe to hybridize a filter displaying multiple gene targets, an approach termed parallel screening for insertion elements, which allows the simultaneous screening for insertions in all genes of an organism, appearing in a population of insertion mutants.

### CROSS-REFERENCE TO RELATED APPLICATIONS

This application is a continuation of application Ser. No. 09/578,361, filed May 24, 2000, pending, the contents of the entirety of which are incorporated herein by this reference, which is a continuation of PCT/EP98/07551 filed Nov. 23, 1998, designating the United States of America.

### TECHNICAL FIELD

The current invention relates to a method of parallel and, as a consequence thereof, simultaneous screening for one or more gene insertion mutants in a population of any organism.

### BACKGROUND

Gene disruption is a powerful tool to assign biological functions to the proteins that are encoded by, for example, the numerous uncharacterized open reading frames (“ORFs”) resulting from genome projects or expressed sequence tags (“EST”) databases. To characterize the biological function of these ORFs efficiently, three problems must be addressed: (i) how to obtain a saturated population of mutants; (ii) how to efficiently identify the genes that have been mutated in the different individuals or, vice versa, how to find individuals mutated in a specific gene; and (iii) how to analyze their phenotypes.

Several strategies have been reported to disrupt specific genes or to simplify the identification of disrupted ORFs. In Saccharomyces cerevisiae, tagged mutants have been generated en masse by using random transposition of the endogenous Ty1 element (Smith et al., 1995). Alternatively, a yeast library in E. coli was mutagenized with a Tn3 element (carrying lacZ for the monitoring of expression) followed by homologous recombination of the mutated yeast sequences into the yeast genome (Burns et al., 1994). Shoemaker et al. (1996) described an approach to perform a quantitative phenotypic assay of yeast deletion mutants, consisting of three steps: (i) replacement of each of the ˜6000 ORFs of S. cerevisiae with a unique 20 bp tag and a kanamycin marker by homologous recombination; (ii) pooling of the deletion mutants and competitive growth under different selection conditions; (iii) amplification of the tags of the surviving strain and identification of their corresponding genes by hybridization of the isolated tags to a micro array displaying oligonucleotides complementary to the tags for all the yeast genes.

For other organisms, it is not yet possible or too laborious to obtain mutants through homologous recombination. Instead, large populations of individuals carrying random transposon insertions have been generated for Drosophila melagonaster (Ballinger and Benzer, 1989, Kaiser and Goodwin, 1990), Caenorhabditis elegans (Zwaal et al., 1993), Petunia hybrida (Koes et al., 1995) and Zea mays (Das and Martienssen, 1995). For Arabidopsis thaliana, large populations of T-DNA insertion mutants have been obtained through Agrobacterium mediated transformation (Bechtold et al., 1993, Mc Kinney et al., 1995, Mollier et al., 1995). A polymerase chain reaction (“PCR”)-based approach is used to screen these populations for individuals harboring insertions in genes of interest. By combining a gene-specific and an insertion-specific primer in the PCR screen, specific products are amplified only when the transposable element has inserted in that gene (Ballinger and Benzer, 1990, Kaiser and Goodwin, 1990). These populations are commonly organized in pools in a pyramidal or 3D fashion, to allow fast screening and easy identification of the individuals carrying a mutant allele.

Using such an approach, the PCR screen has to be repeated for each individual ORF, or at the most, a limited set of gene-specific primers can be combined in the same screen (Mc Kinney et al., 1995). This is a time consuming process and is extremely laborious to carry out.

### SUMMARY OF THE INVENTION

The method according to the invention offers an attractive alternative to the existing strategy used to screen large populations for insertion mutants. The rationale of the previously described PCR-based screening for insertion mutants is that whenever an insertion element has inserted in a gene, a product can be amplified in a PCR reaction using a gene- and an insertion-specific primer (Ballinger and Benzer, 1989; Kaiser and Goodwin, 1990).

The technique according to the invention reproducibly amplifies all the transposon flanking sequences in a pool of individuals, and subsequently identifies insertions by hybridization with a gene-specific probe. By eliminating the use of the gene-specific primer, it becomes possible to perform a serial screening for insertions in a large set of genes using a single set of PCR reactions. Alternatively, the flanking sequence pools can be used as probes to screen target genes.

This approach was tested using nested iPCR as a means to amplify the transposon dTph1-flanking sequences from the DNA pools of a 3D library of 1000 W137 Petunia individuals. To amplify dTph1-flanking sequences a tetracutter was used that does not cut within the element, the obtained DNA fragments were circularized, dTph1 flanking sequences were amplified by iPCR using a set of internal primers, and a single primer based on the TIR for nested reamplification. The results indicate that a dTph1 insertion in a specific gene of a specific plant can be detected against a background of about hundred wild type individuals, both in the serial and in the parallel screening method (see below). The variation in the signals produced for the positive control insertion in PhAp2A however indicate that the protocol had to be optimized to ensure that the dTph1 flanking sequences of all the pool samples representing the population are sufficiently well amplified. The efficiency of the tagging system will influence the optimal size of the population to be screened. If n insertions per 1000 plants are found for a specific cDNA clone, 3n signals will arise. Direct identification of the candidate plant is only possible if n equals one. Somatic insertions into a gene will interfere and may lead to background hybridization. Stable insertion systems (for example T-DNA insertions) or systems with a controllable or low somatic insertion frequency will not suffer this problem.

The choice of the targets may also influence the efficiency of recovery of phenotypic mutants. If only cDNA targets are used, the percentage of intron and promoter amplified fragments that are recovered should statistically go down, and more knock-out mutants may be recovered, especially if the transposable element is small, harbors no transcription termination signals, and is unlikely to interfere with the gene function when it has inserted in an intron, like dTph1 in Petunia.

The presented technique can be applied to the respective insertion mutagenesis system used for any organism. When the tetracutter recognizes a site within the insertion element, as it will in most cases, a set of two insertion-specific primers must be used to amplify the flanking sequences. Instead of iPCR, the transposon flanking sequences can also be obtained by adaptor mediated PCR, as described by Van Den Broeck et al., (1998) or by vectorette PCR (Riley et al., 1990). The filter-based screening, described here to illustrate the potential of the technique, could readily be extended to the microarray technology. In theory, microarrays can be produced displaying up to and over 20,000 targets, and hybridization of fluorescently labeled flanking sequences with those targets can be detected by means of confocal microscopy (Schena et al., 1995). When (micro)arrays, orderly representing the whole genome of an organism are available, the method could be used to quickly catalogue the individual insertions and simultaneously assess the randomness of the insertions in a given population. Micro-arrays containing cDNA targets of an organism can also be used to screen for tagged genes in a closely related organism that is not in itself the subject of major sequencing efforts, if the stringency conditions used in the hybridization reactions are adapted. Such catalogues could also be helpful in identifying genes in map-based cloning strategies. After fine mapping of an interesting mutation, seeds of all the different insertion mutants in the region of interest could be retrieved from the insertion library. Mutant phenotypes in the progenies of the primary insertion mutants could then be compared to that of the originally mapped mutant. When the same or a comparable phenotype arises in such a family, the gene of interest has probably been tagged and thus been identified. Catalogues of insertion mutants can also be generated by isolation and sequencing all the insertion flanking sites found in a population. Using an efficient gene- and enhancer-trap transposon mutagenesis system, based on the maize elements Ac/Ds and the GUS reporter gene, a population of more than 10,000 single copy insertions mutants in Arabidopsis has been generated and a few hundred of these insertion alleles have been sequenced. The dTph1 element in Petunia has a high copy number (150-200) in the line used for tagging. Every W137 individual harbors ten to twenty new germline insertions. Analysis of the new insertion sites would require cloning of the individual dTph1 flanking sequences and selection of the 10% of clones that represent novel insertions. For high copy number tagging systems like dTph1 in Petunia, Mu in maize, and Tc1 in C. elegans, the sequencing approach therefore seems too laborious.

The current screening system according to the invention offers the opportunity to screen for insertion mutants in large populations of individuals. When used at its full potential, the efficiency of screening will be increased several orders of magnitude over that of the existing PCR-based screening method.

So the first aspect of the current invention is a method of simultaneously screening for one or more gene insertion mutants in a population of any organism by:
a) preparing an insertion element mutant library originating from a defined population of an organism or cell line wherein said insertion(s) have to be detected;
b) amplifying the insertion element flanking sequences from said insertion element mutant library; and
c1) either fixing the set of thus obtained nucleic acid amplification products representing said insertion element flanking sequences derived from said insertion element mutant library to a solid support as target for hybridization; or
c2) producing a set of labeled amplification products representing said insertion element flanking sequences derived from said insertion element mutant library to use as probe to hybridize to a solid support to which one or more nucleic acids have been fixed as target(s) for hybridization.

The thus obtained nucleic acid amplification products in above captioned step b) can, for example, be obtained by iPCR using at least one primer or a set of primers based on the sequence of the insertion element or by Transposon Display PCR.

Above-mentioned iPCR is performed by:
a) digesting the nucleic acid sequences of said insertion element mutant library with a restriction enzyme which optionally recognizes motifs of four nucleotides in the genomic DNA, or with a combination of restriction enzymes resulting in a collection of amplifiable fragments;
b) self ligation of the genomic fragments thus obtained; and either
c1) amplification of insertion element flanking sequences using a set of internal primers; or
c2) amplification of insertion element flanking sequences using a primer or a set of primers based on the terminal part of the insertion element.

The above described amplification products of step c1 can be re-amplified using at least one primer or a set of two nested primers based on the sequence of the insertion element.

The above-described amplification products in step b) can also be obtained by so-called transposon display amplification whereby said transposon display amplification is performed by:
a) digesting the nucleic acid sequences of said insertion element mutant library with a first restriction enzyme that recognizes six conserved nucleotides in the insertion element and with a second restriction enzyme that recognizes a motif of four nucleotides in the genome generating at least one restriction fragment per insertion containing at least the hexacutter site, a part of the insertion element, and part of the insertion element flanking sequence;
b) ligation of a biotinylated adaptor to the hexacutter sites and a ligation of a second adaptor to the tetracutter sites of the restriction fragments generated in a);
c) selection of biotinylated restriction fragments using magnetic streptavidin beads;
d) amplification of insertion element flanking sequences using a primer based on the sequence of the biotinylated adaptor and on the insertion element sequence and a primer complementary to the second adaptor; and
e) re-amplification of said insertion element flanking sequences using a nested primer based on the insertion element and a primer complementary to the second adaptor.

The solid support as described above in any of the methods can be a filter, micro-array, microchip containing nucleic acid sequences, or a bead and the like, whereas the nucleic acid sequence is genomic DNA, cDNA, oligonucleotide sequence or PNA.

In the method according to the invention an insertion element mutant library may comprise 30 DNA samples from 100 plants each wherein the insertion element mutant library is built in a 3D array of 10 Block, 10 Row and 10 Column pools, each containing DNA of 100 plants characterized by the three coordinates B, R, C.

Depending on which method according to the current invention is used, BfaI or MseI and/or MunI are used as restriction enzymes.

The organism, having a genome wherein one or more gene insertions according to the invention has to be detected, can in principle be any organism including micro-organisms, such as yeast, C. elegans, monocytelydoneous or dicotyledoneous plants, such as preferably maize, but also Arabidopsis thaliana, Petunia, and other flowering plant species and the like.

A second aspect of the present invention is a composition of elements in the form of a test kit for performing any of the methods described above wherein said kit comprises at least an organized genomic DNA library and, optionally, a restriction enzyme such as BfaI capable of recognizing four nucleotides in the genome.

The kit may also be composed of one or more filters carrying an array of amplified products of the flanking sequences derived according to the current invention from a defined organized population of any organism.

So part of the present invention is a kit for performing any of the methods of the invention, comprising at least DNA samples of an insertion element mutant library and optionally, a set of restriction enzymes and/or primers.

For performing any of the methods of the invention the kit may comprise at least a set of amplified insertion element flanking sequences wherein said set of insertion element flanking sequences optionally have been fixed on a solid support such as a filter, micro-array, microchip containing nucleic acid sequences, or a bead, whereas said set of insertion element flanking sequences are either present in soluble form or dried form.

The set of insertion element flanking sequences can optionally be labeled with for instance fluorescein, however any suitable label known to a person skilled in the art can be used for this purpose.

The system wherein the organized genomic DNA library according to the invention is present, can be, although not limited to, a so-called three dimensional system (3-D array as depicted in FIG. 1A) and is clearly described in Koes at al. (1995) being herewith incorporated by reference.

### BRIEF DESCRIPTION OF THE FIGURES

FIG. 1A depicts a 3-D array showing amplification of pools of dTph1 flanking sequences.

FIG. 1B graphically depicts the simple screening for insertion mutants.

FIG. 1C graphically depicts parallel screening for insertion mutants.

FIG. 2 depicts the amplification and detection of transposon flanking sequences using transposon display.

FIG. 3 depicts the generation of a 3D library of flanking sequences.

FIG. 4a depicts screening with 4 Kb Xbal Ap2-A fragment as a probe. Transposon display fragments spotted: A-G represent a dilution series as described herein, while Wt is wild-type. The probe was fluorescein-labeled, with an exposure time of 5 minutes.

FIG. 4b depicts parallel screening for Stig 1 insertion mutant. 49 plants (left side) and 100 plants (right side) were pooled and sampled. Two samples (Block III and Row 4) were known to contain an insertion mutant for the Stig 1 gene, whereas one sample (Block I) was known not to contain this insert. Six replicate filters were prepared, harboring spot-blotted DNA from the Ap2A gene (left side of each filter) or the Stig 1 gene (right side). The samples identified did not contain an insertion for the Ap2A gene. PCR products from each sample, obtained by Transposon Display, were fluorescein-labeled and used as a probe.

### DETAILED DESCRIPTION OF THE INVENTION

The current invention is hereunder described in more detail for sake of clarity to get a better understanding of the invention. In addition, some definitions are hereunder given of what is meant by terms used in the current description.

Definitions

“Restriction endonuclease” or “restriction enzyme” is an enzyme that recognizes a specific nucleotide sequence (target site) in a double-stranded DNA molecule, and will cleave both strands of the DNA molecule at every target site. The cleavage of the DNA molecule can appear at different positions on the two strands, resulting in the generation of single-stranded DNA overhangs, which are specific for the restriction endonuclease.

“Restriction fragments” are DNA molecules produced by digestion with a restriction enzyme. Any given genome will be digested by a particular restriction enzyme into a discrete set of restriction fragments.

“Insertion element” refers to any nucleic acid sequence integrated in a genome. Insertion elements comprise, by way of example, but are not limited to, transposable elements, retrotransposons, and T-DNA insertions.

“Insertion element flanking sequences” are the nucleotide sequences flanking a given insertion element in the genome. Insertion flanking sequences can be amplified, by way of non-limiting example, using inverse PCR (iPCR), Transposon Display, Amplification of Insertion Mutagenized Sites (AIMS), vectorette PCR, or thermally asymmetric interlaced PCR (TAIL-PCR).

“Insertion element mutant library” in this invention refers to a set of DNA samples isolated from and representing the population. The DNA pools are organized in a way that allows reference to the individual or cell line in which an insertion element has inserted in a defined position in the genome, when an insertion is detected in a specific subset of DNA pools of the insertion element mutant library. By way of example, the insertion element mutant library representing a population of 1000 individuals may be obtained by giving each of the 1000 individuals three coordinates (B, R, C) with (1<=B<=10, 1<=R<=10, 1<=C<=10), and to isolate 30 DNA samples containing the DNA of the 100 individuals, in which each DNA sample is derived from the 100 individuals for which one of the three coordinates equaled a number between 1 and 10.

“Ligation” is the joining of the phosphorylated 5′ end of a nucleic acid to a 3′ end of a nucleic acid. Ligation of double-stranded DNA is mediated by the enzyme DNA ligase and involves the joining of the 5′ and the 3′ end of a DNA molecule to the 3′ and 5′ end a DNA molecule. The 5′ end of a nucleic acid can be joined to a 3′ end of a second nucleic acid in an intermolecular reaction, or it can be joined to its own 3′ end in an intramolecular reaction which will be referred to as back-ligation or self-ligation.

“PCR” refers to enzymatic reaction wherein DNA fragments are synthesized from a substrate DNA in vitro as described by Cetus in U.S. Pat. Nos. 4,683,195 and 4,683,202. Briefly, the reaction involves the use of two synthetic oligonucleotides/primers, which are complementary to nucleotide sequences in DNA molecules which are separated by a short distance of a few hundred to a few thousand base pairs, and the use of a thermostable DNA polymerase. After denaturing double-stranded DNA molecules, the primers will anneal to the complementary nucleotides in the template molecule at a specific annealing temperature. A suitable DNA polymerase then initiates the synthesis of a DNA strand complementary to the template molecule. The chain reaction continues for a variable number of cycles, for instance, a series of 10 to 30 cycles.

“DNA amplification” is used to denote the in vitro synthesis of double-stranded DNA molecules using, for instance, PCR as nucleic acid sequence amplification technique. The products of the PCR are called amplified DNA fragments.

“Primer,” in general, refers to a DNA strand which can prime DNA synthesis. DNA polymerase cannot synthesize DNA de novo without primers.

An “adaptor” is a double-stranded oligonucleotide that contains a single-stranded DNA overhang that can be joined by ligation to a restriction fragment that contains a complementary DNA overhang.

“Nucleic acids” comprise DNA, RNA, or synthetical analogues thereof. The genome of an organism consists of deoxyribonucleic acid (DNA). DNA is a double helix consisting of a two chains of nucleotides. Each chain consists of a strand of nucleotides linked to each other via phosphodiester bridges between the 5′ hydroxyl group of the sugar of one nucleotide linked to the 3′ hydroxyl group of the sugar of the adjacent nucleotide. As a consequence, a single-stranded DNA molecule has an orientation defined by its 5′ and its 3′ ends. DNA duplexes contain two single-stranded DNA molecules joined together in the opposite orientation. Single-stranded DNA consists of a combination of four different nucleotides, containing the bases adenine (A), guanine (G), thymine (T), and cytosine (C). The DNA duplex is a result of the formation of hydrogen bridges between guanine and cytosine and between adenine and thymine residues located on the two different strands. Guanine and cytosine are complementary bases, as are thymine and adenine. The information contained within the genome is transcribed into RNA. RNA is a single-stranded chain of ribonucleic acid. RNA consists of four different nucleotides, containing the bases adenine, guanine, cytosine, and uracil (U). Uracil and adenine are complementary bases.

“Hybridization” is the process of the binding of a nucleic acid to a second nucleic acid through the formation of hydrogen bridges between the bases of the nucleic acids. Hybridization of nucleic acids is obtained by interaction of two complementary nucleic acids at specific conditions of ionic strength, pH, and temperature.

“Denaturation” is the process of separation of two nucleic acid strands. Denaturation can be obtained by increasing the temperature or lowering the ionic strength of a double-stranded nucleic acid solution.

A “single-stranded nucleic acid” is complementary to a second single-stranded nucleic acid in case the two strands contain the complementary nucleotides in the opposite order. A nucleic acid can hybridize to a molecule which is its perfect complement, to a nucleic acid which comprises its perfect complement, or to a nucleic acid which is comprised in its perfect complement. Under tolerant hybridization conditions, a nucleic acid can also hybridize to non-perfect complements, provided the non-perfect complement has a sufficient amount of similarity with the perfect complement. Nucleic acids which are not identical but which exhibit a significant amount of similarity are called homologous.

The term “probe” refers to a single-stranded nucleic acid which is capable of binding to a second nucleic acid, which is called the target. Typically, the probe is associated with a label to identify the target to which the probe binds.

The term “label” refers to a molecular moiety capable of detection and may include, by way of example, radioactive isotopes, chemiluminescence, fluorescence, dyes, etc. A label can also be a moiety which is capable of interacting with other molecules, which can be detected directly or indirectly, such as, by way of example, but not limited to, antigens which can be detected immunologically by means of antibodies coupled to enzymes which can convert a substrate into a detectable molecule.

In this invention, the term “probe” comprises the nucleic acid or the pool of nucleic acids which is hybridized to the target, the probe being a nucleic acid or a pool of nucleic acids in solution, and the target being a nucleic acid or a set of nucleic acids fixed to a solid support. “Solid support” refers to conventional supports comprising filters, membranes, silicate supports, such as glass, but also refers to more sophisticated supports like micro-arrays or microchips containing nucleic acid sequences.

The term “population” comprises a group of discrete individuals or a group of discrete cell lines, a group containing at least one but typically 1000 individuals or cell lines.

More Detailed Outline of the Technique

To screen for insertion mutants in an efficient way, the plants are not analyzed individually but organized for example in a 3D array of Block, Row, and Column pools of 100 plants as described by Koes et al. (1995). In a population of 1000 plants, each individual is characterized by 3 coordinates (B_{1≦B≦10}, R_{1≦R≦10}, C_{1≦C≦10}).

Ten Block, ten Row, and ten Column pools of DNA are prepared of 100 plants each generating an insertion element mutant library consisting of 30 DNA samples.

Screening for Insertion Mutants Using iPCR and/or Transposon Display

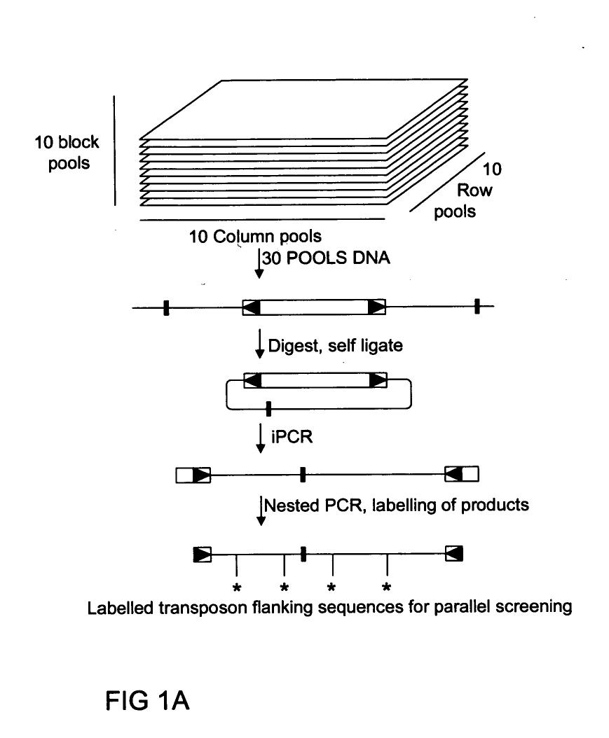

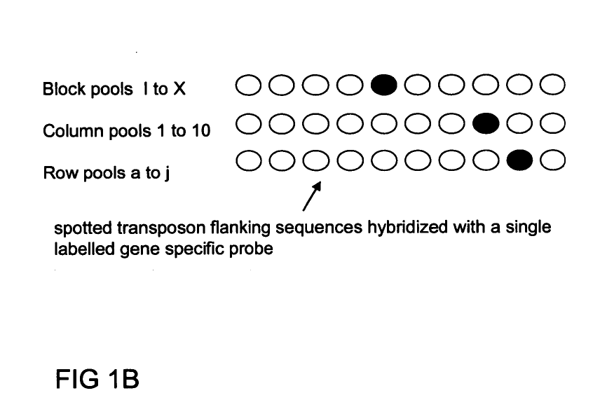

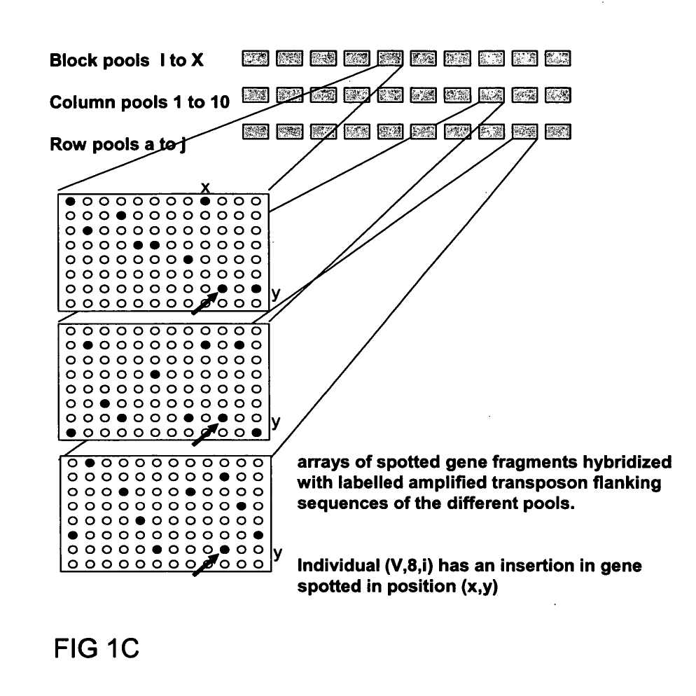

The insertion element flanking sequences in the different samples of the insertion element mutant library are recovered in four steps. Genomic DNA is digested using a tetracutter restriction enzyme. The obtained fragments are circularized by self-ligation, amplified using a set of internal primers derived from the insertion element, and re-amplified by nested PCR to ensure the specificity of the obtained products (FIG. 1A).

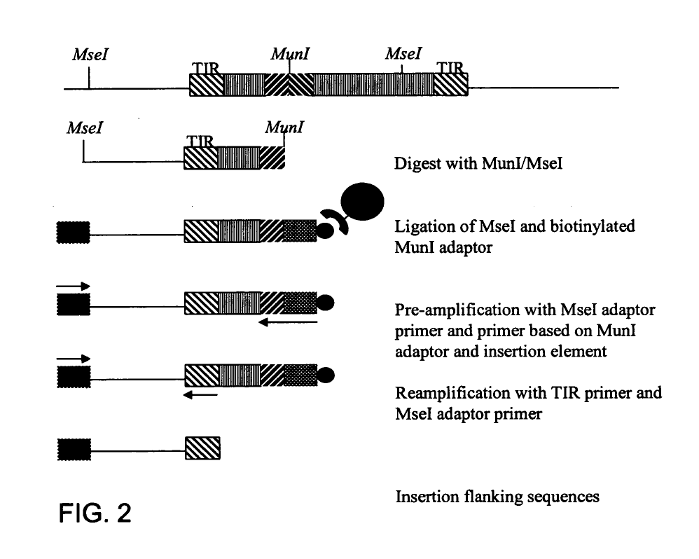

Alternatively, the sequences flanking the insertion elements in a specific pool can be recovered using TRANSPOSON DISPLAY amplification. In this case, the DNA samples of the insertion elements mutant library are digested with a restriction enzyme recognizing a site of 6 bases (this restriction enzyme is referred to as the hexacutter) conserved in the insertion element and a restriction enzyme recognizing a site of 4 bases (this restriction enzyme is referred to as the tetracutter). Adaptors are ligated to the obtained fragments and those fragments containing the hexacutter site are selected. Insertion flanking sequences can be amplified in two steps. In the pre-amplification reaction, a primer based on the hexacutter site adaptor and the insertion element is used in combination with a primer based on the tetracutter site adaptor to amplify a subset of PCR fragments. In the second step, these fragments are used as a template to amplify insertion-specific fragments using a transposon-specific primer and a primer based on the tetracutter site adaptor (FIG. 2).

To screen for individuals carrying insertions in specific genes of interest, two approaches are possible.

In the first approach, the 30 amplified flanking sequence pools can be displayed on filters and hybridized with one target gene of interest at a time (FIG. 1B). This method already offers an advantage over the existing gene-specific primer-based methods, since the same PCR products can be used to screen many, at least 300-500, genes while upscaling is possible. In the second approach, a large set of targets is displayed on 30 replica filters, and hybridized with the labeled set of 30 amplified sequence pools (FIG. 1C). This approach becomes useful when more than 30 target genes are being screened for. One great advantage of this method is that a priori no sequence information of target genes is needed.

Determining the Detection Limits

A reconstruction experiment was performed in which Petunia W137 genomic DNA was mixed with decreasing amounts of DNA of the heterozygous insertion mutant PhAp2A (V2025-6) and subjected to the iPCR protocol. The iPCR products were blotted on mini filters and hybridized to a range of dilutions of an Ap2A-specific probe. At a probe concentration of 250 μg/ml the hybridization signal of the 1/256 PhAp2A(V2025-6)/W137 dilution could clearly be detected above the background signal. Since Petunia hybrida W137 individuals harbor around 150-200 dTph1 elements, this means that a specific transposon flanking sequence was detected against a background of ˜50000 (256×200) copies. Consequently, specific insertions should be detectable in pools of 100 W137 Petunia plants. For other model systems, where less insertions are to be expected per individual, bigger pools could be screened.

Construction of a 3D Insertion Flanking Sequence Library and Screening for Insertion Mutants

Thirty genomic DNA samples were prepared of pools of a hundred individuals each, representing a population of 1000 W137 plants. An individual plant carrying the PhAp2A(V2025-6) insertion allele was included in the population to serve as a positive control. The flanking sequences of the 30 DNA pools and an insertion mutant dilution series were generated using iPCR with the terminal inverted repeat primers of the transposon, with a set of internal transposon primers or combining the latter iPCR reaction with a nested reamplification with the terminal inverted repeat primers of the transposon (compare FIG. 1A). The last approach clearly gave the best amplification on the pools.

For the technique to work, it is essential that all pools have efficiently amplified the flanking sequences of the dTph1 elements. To control the general quality of the amplification reactions, we checked whether two dTph1 flanking fragments, Pete1 and Pete2, had been amplified equally in all pools. Pete1 and Pete2 were identified by Transposon Display (Van den Broeck et al., 1998) and are inherited by all the individuals of the W137 population. This implies that they are present at a 200 fold higher concentration in the pools than a heterozygous insertion present in a single plant of the population. All pools were found positive for Pete1 and Pete2, except R9 in the first series and R5 in the second series.

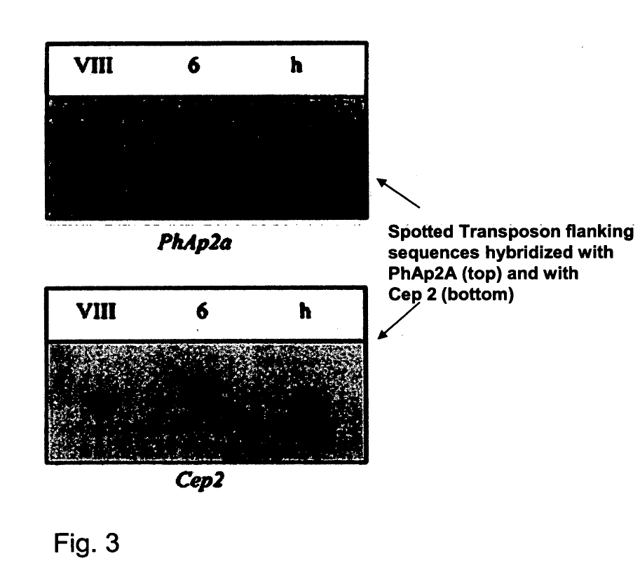

However, the flanking sequences of dTph1 in Cep2 in plant (VIII, 6, h) of this population, previously identified by the classical 3D screening approach, did only show up as a positive in one of the three dimensions. Therefore, a more sensitive positive control was developed by adding a 1/100 dilution of genomic DNA of a plant heterozygous for the PhAp2A (V2025-6) insertion allele to all of the pools of genomic DNA. After digestion and nested iPCR, the pool products were spot blotted and hybridized with a PhAp2A-specific probe. The protocol was repeated until all pools proved positive for the PhAp2A insertion. After this qualitative improvement, the Cep2 insertion could be detected in the pools that we expected to be positive (FIGS. 3A and 3B).

Parallel Screening for Insertion Mutants

For parallel screening, the transposon flanking sequence pools were labeled and used as a probe to hybridize potentially tagged genes. A dilution series of PhAp2A and negative control target DNA (vector sequence) was spotted on small round filters and hybridized with a labeled Block III pool to determine the amount of DNA that needed to be displayed on the target filters to obtain a clear positive signal. A set of 30 replicas, each displaying 80 target genes, was made using a Beckman Biomek 2000 Laboratory Automated Workstation. Most of the displayed targets (73) were PCR amplified inserts from random picked cDNA clones from a carpel-specific cDNA library. Among the seven targets with known sequences was PhAp2A, which was included as a constitutive positive control for the 30 samples. Using the classical 3D screening method, a dTph1 insertion had been identified in PhAp2C in plant (V, 8, g), thus PhAp2C was used as a positive control representing a single insertion mutant appearing in the population.

The 30 pools of transposon flanking sequences obtained by nested iPCR were fluorescein labeled and hybridized to the 30 target gene replicas. Each target was displayed three times and formed a pattern which can be easily distinguished from spurious spots on the filter. The control PhAp2A signal can be detected as a three dot diagonal at position (2, E) on each filter using probes made of the insertion element flanking sequences which had been amplified nicely as determined by the quality control assay described under construction of a 3D insertion flanking sequence library. A dTph1 insertion in PhAp2C was detected in the correct position at the diagonal of position (4, E) of the filters hybridized with the probes made of the insertion element flanking sequences which passed the quality control assay, and which were derived from the insertion element mutant library samples which contained the DNA of the individual plant that contained a dTph1 insertion in PhAp2C.

### EXAMPLES

Method for Determination of Detection Limits

Genomic DNA of the heterozygous PhAp2A insertion mutant was diluted with increasing amounts of wild type DNA (ranging from 1/1 to 1/256 insertion mutant/wild type DNA). 10 μg of each DNA mix was digested in 100 μl 1× New England Biolabs buffer 4 with the tetracutter enzyme BfaI, which does not cut within the 284 bp dTph1 element. After complete digestion, the enzyme was heat inactivated and the mixture was phenol:chloroform extracted, precipitated and dissolved in dd H₂O. 2 μg of DNA was ligated ON at 14° C. in 400 μl 1×T4 ligation buffer in the presence of 2.5 units of T4 DNA ligase. The ligation mixture was extracted with phenol:chloroform, with chloroform and precipitated in the presence of 20 μg of calf thymus tRNA, washed and dried. The vacuum dried pellet was re-suspended in 30 μl of dd H₂O. 5 μl of the self-ligated fragments were used in the iPCR reaction with the outward transposon inverted repeat primer (TIR), consisting of 35 cycles of 1 minute denaturation at 95° C., 1 minute annealing at 55° C., and 1.5 minute polymerization at 72° C. The PCR reactions were also performed on equivalent amounts of undigested W137 and insertion mutant DNA. The reaction products were spotted in 8-plicate on individual Hybond N⁺ filters that had been cut small enough to fit into 96 well plates, denatured, neutralized and baked as prescribed by the manufacturer. The filters were pre-hybridized in 50 μl of hybridization mix (as prescribed by Amersham). 50 ng of a 900 bp SpeI/EcoRI Ap2A fragment was ³²P labeled with a Megaprime kit in a 50 μl reaction with 5 μl α-dCTP (incorporation˜50%). Half of the denatured probe was diluted to 1200 μl with hybridization mix, 600 μl was divided in 12 aliquots of 50 μl, that were added to the pre-hybridization mix, 600 μl was used for seven consecutive two-fold dilutions. Thus, the concentration of the probe in the first series of 12 hybridizations was ˜1 ng/100 μl, decreasing each time by a factor of two for the next seven dilutions. Washes were performed at 60° C., 2× in 2×SSC, 0.1% SDS and 4× in 0.5×SSC, 0.1% SDS for 10 minutes each. Hybridized filters were exposed to a Phosphor Image cassette and scanned using a Beckman Phosphor Imager.

Method for the Construction of a 3D Insertion Flanking Sequence Library Including Quality Control

1000 W137 plants were organized in a 3D array of 10 blocks, 10 rows, and 10 columns, as described by Koes et al. (1995). DNA was extracted from the leaves of the 30 pools using a CTAB extraction method. DNA extracted from a heterozygous Ap2A insertion mutant was added to each pool at a ratio of 1:100 as a positive control. 10 μg of each of the 30 DNA samples was digested at 37° C. for 6 hours by 10 units of Biolabs BfaI restriction enzyme in 1×NEB4 buffer. The restriction reactions were extracted with phenol, phenol:chloroform, and chloroform. 1 μl of 10 mg/ml tRNA was added, the samples were precipitated with NaOAc and EtOH, washed with 70% EtOH and dissolved in 100 μl H₂O. 20 μl of this mix (2 μg) was ligated overnight at 16° C. by 6 units of T4 DNA ligase (Pharmacia) in 400 μl of ligation buffer (10 mM MgCl2, 30 mM Tris.Cl pH 7.8, 10 mM DTT, 0.5 mM ATP). The ligation mixture was extracted with phenol, phenol:chloroform, and chloroform, precipitated as above, and dissolved in 30 μl H₂O. 5 μl of this solution was used as template in a first amplification reaction performed with two internal transposon-specific primers (IntA: 5′-GGGAATTCTTGAACGAGTTGTCCTC-3′ (SEQ ID NO:1) and IntB: 5′-GGGAATTCAGTGTAAATTTTGCGC-3′ (SEQ ID NO:2), see also Souer et al., 1995), for 35 cycles of 30 seconds at 94° C., 30 seconds at 55° C. and 1 minute 30 seconds at 72° C. in 50 μl 1× Perkin Elmer with 30 pmol of each primer, 200 nM dNTPs, 4 mM MgCl2 and 1 unit Perkin Elmer Taq polymerase. 3 μl of the resulting transposon flanking sequences were re-amplified with a primer based on the terminal inverted repeat of the transposon (5′-GGGAATTCGCTCCGCCCCTG-3′) (SEQ ID NO:3).

Quality Control of the Amplified Flanking Sequence Pools

0.25 μl of the PCR products were spot blotted on a Hybond N⁺ membrane and hybridized with an α-³²P dCTP Ap2A-specific probe to see whether the control insertion mutant could be detected. Washes and detection were as in the reconstruction experiment.

Method for the Parallel Screening of Insertion Mutants

20 μl aliquots of target DNA solution (100 ng/μl) were distributed in V bottom shaped microtiter plates and spotted on Hybond N⁺ filters using a Beckman Biomek 2000 Laboratory Automation Workstation. Each replica had the size of 1/3 96 well microtiterplate. On each of the 4×8 positions (from 1 to 4 and from A to H), a matrix of 3 targets was displayed as follows:

$$\begin{matrix}a & b & c \\ a & c & b \\ c & a & b\end{matrix}$$

In this way, 4×8×3=96 targets can be displayed per replica (we have used a 4×7×3=84 target array). The filters were treated for hybridization as described by Amersham. 5-10 μl of the amplified flanking sequence pools were labeled with fluorescein using the Gene Images random prime labeling module (Amersham). A small amount of IR primer was added in the labeling reaction to ensure that small fragments would be labeled. The replica filters were hybridized in 5 ml hybridization buffer and washed according to the Amersham protocol. The hybridizing signals were visualized using the Gene Images Detection Module (Amersham) after exposure to Fuji X-RAY films.

Screening for Insertion Mutants Using Transposon Display

Determining the Detection Limits

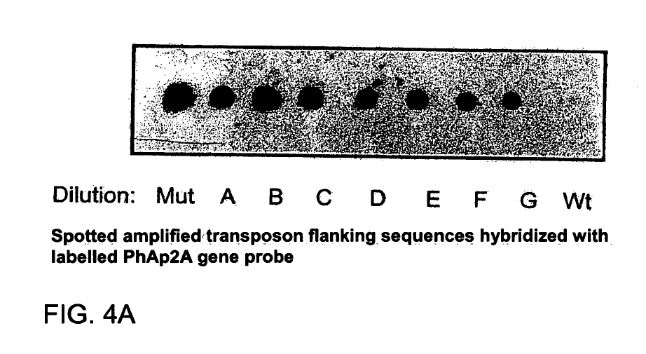

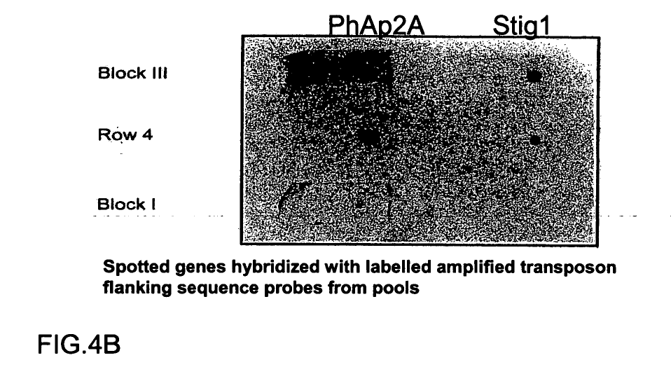

A reconstruction experiment was performed in which Petunia W137 genomic DNA was mixed with decreasing amounts of DNA of an insertion mutant containing a dTph1 insertion in Ap2A (A=1/2, B=1/4, C=1/8, D=1/16, E=1/32, F=1/64, G=1/128), and subjected to the transposon display amplification protocol, which was performed with unlabeled PCR primers. The Transposon Display amplification products were spot blotted on filters and hybridized with a labeled Ap2A-specific probe. The amplified dTph1 flanking fragment containing part of the Ap2A gene could readily be detected in the 1/128 dilution of the mutant DNA with the wild type W137 DNA (FIG. 4A).

Construction of a 3D Library of Insertion Flanking Sequences and Screening for Insertion Mutants.

A second reconstruction experiment was performed in which the genomic DNA of a pool of respectively 4 (2×2), 9 (3×3), 16 (4×4), 25 (5×5), 36 (6×6), 49 (7×7), 64 (8×8), 81 (9×9) and 100 (10×10) different individuals, was isolated. In each pool, one of the individuals was subsequently replaced by an individual containing a dTph1 insertion in the Ap2A gene and DNAs were extracted.

Transposon Display amplification was performed on each of the pools using unlabelled primers and the resulting Transposon Display amplification products were spot blotted on filters and hybridized with an Ap2A-specific probe. The amplified dTph1 flanking fragment containing part of the Ap2A gene could readily be detected as a positive signal in each of the pools harboring the Ap2A insertion. In control pools not containing DNA from the insertion mutant, no such signal was observed. The dTph1 flanking sequences could then be amplified from all 30 pools of a population of 1000 organized in Block, Rows and Columns, spotted on a filter and used to screen for insertions in any gene of interest.

Parallel Screening for Insertion Mutants

In order to screen for insertions in many genes simultaneously, the amplified Transposon Display fragments are labeled and used as a probe to hybridize a filter containing a set of target genes.

Both Transposon Display fragments obtained from the amplification of dTph1 flanking fragments from the dilution series F and G, described above, and those obtained from the 2×2, 6×6, 8×8 and 10×10 pools containing an individual with a dTph1 insertion in Ap2A, mentioned above, were separately labeled and used to hybridize filters displaying a fragment of the Ap2A gene. All probes obtained by Transposon Display PCR on the pools containing the Ap2A insertion, were able to detect the Ap2A gene on the filters.

The dTph1 flanking sequences can be amplified from all 30 pools of a population of 1000 organized in Block, Row and Columns, labeled, and used as probes to hybridize filters containing multiple gene targets. An insertion in gene Stig1, was identified in the correct pools of Transposon Display fragments obtained from such a population (FIG. 5B).

Methods to Screen for Insertion Mutants Using Transposon Display

Transposon Display was performed as described by Van den Broeck et al., 1998, with the following modifications:

The streptavidin beads were suspended in 50 instead of 200 μl T01E buffer, the selective pre-amplification reactions were performed with 5 μl of the above 50 μl in a reaction volume of 50 μl using the MunI-ACAC primer and an MseI primer, using however a touch down PCR profile as described in the hot PCR. The pre-amplification products were diluted 10 times and re-amplified using 5 μl of the diluted pre-amplification product, the IR primer and the MseI primer, in a reaction volume of 50 μl but without labeling the IR primer, resulting in/the final product.

To perform simple screening for insertion mutants, the 50 μl of final product was dried, re-suspended in 5 μl water, denatured and spotted on Hybond N⁺, and hybridized with a denatured gene-specific probe labeled using the CDP-star kit of Amersham, in a buffer containing 5×SSC, 60° C., and blots were washed at 60° C. at a stringency of 0.5×SSC.

To perform parallel screening for insertion mutants, 2 μl of the 50 μl of the final product was labeled using a linear PCR using 1.5 μl IR primer, 10 μl nucleotide mix of the CDP-star kit of Amersham, 5 μl 10×PCR buffer and 0.2 μl Taq polymerase in a volume of 50 μl, using a PCR profile of 30 cycles of 94° C./30 seconds, 56° C./30 seconds, 72° C./60 seconds.

Hybridizations of filters with target genes were performed with these probes in a buffer containing 5×SSC at 60° C., and blots were washed at 60° C. at a stringency of 0.5×SSC.

### References

Ballinger, D. G., and Benzer, S. (1989). Targeted gene mutations in Drosophila. PNAS 86, 9402-9406.

Bechtold N., Ellis, J., Pelletier, G. (1993). In planta Agrobacterium mediated gene transfer by infiltration of adult Arabidopsis thaliana plants. C.12. Acad. Sci. Paris, Sciences de la vie/Life Sciences 316, 1194-1199.

Burns, N., Grimwade, B., Ross-Macdonald, P. B., Choi, E-Y., Finberg, K., Roeder, G. S. and Snyder, M. (1994). Large-scale analysis of gene expression, protein localization, and gene disruption in Saccharomyces cerevisiae. Genes and Dev. 8, 1087-1105.

Das, L. and Martienssen, R. (1995). Site-Selected Transposon Mutagenesis at the hcf106 Locus in Maize. Plant Cell 7(3), 287-294.

Gerats, A. G. M., Beld, M., Huits, H., Prescott, A. (1989). Gene tagging in Petunia hybrida using homologous and heterologous transposable elements. Dev.Gen. 10, 561-568.

Kaiser, K., and Goodwin, S. F. (1990). “Site-selected” mutagenesis of Drosophila. PNAS 87, 1686-1690.

Koes, R., Souer, E., Van Houwelingen, A., Mur, L., Spelt, C., Quattrocchio, F., Wing, J., Oppedijk, B., Ahmed, S., Maes, T., Gerats, T., Hoogeveen, P., Meesters, M., Kloos, D., Mol, J. N. M. (1995). Targeted gene inactivation in petunia by PCR-based selection of transposon insertion mutants. PNAS 92(18), 8149-8153.

McKinney, E. G., Aali, N., Traut, A., Feldmann, K. A., Belostotsky, D. A., McDowell, J. M., Meagher, R. (1995). Sequence-based identification of T-DNA insertion mutations in Arabidopsis: Actin mutants act2-1 and act4-1. Plant Journal 8(4), 613-622.

Mollier, P., Montoro, P., Delarue, M., Bechtold, N., Bellini, C., Pelletier, G. (1995). Promoterless gusA expression in a large number of Arabidopsis thaliana transformants obtained by the in planta infiltration method. C.R.Acad.Sci. Paris, Sciences de Ia vie/Life sciences 318, 465-474.

Riley, J., Butler, R., Ogilvie, D., Finniear, R., Jenner, R., Powell, S., Anand, S., Smith, S., and Markham, A. F. (1990). A novel, rapid method for the isolation of terminal sequences from yeast artificial chromosome (YAC) clones. NAR 18, 2887-2890.

Schena, M., Shalon, D., Davis, R. W. and Brown, P. O. (1995). Quantative monitoring of gene expression patterns with a complementary DNA microarray. Science 270, 467-470.

Shoemaker, D. D., Lashkari, D. A., Morris, D., Mittmann, M. and Davis, R. W. (1996). Quantitative phenotypic analysis of yeast deletion mutants using a highly parallel molecular bar-coding strategy. Nature Genetics 14, 450456.

Smith, V., Botstein, D. and Brown, P. O. (1995). Genetic footprinting: A genomic strategy for determining a gene's function given its sequence. PNAS 92, 6479-6483.

Souer, E., Quattrochio, F., de Vetten, N., Mol, J. and Koes, R. (1995). A general method to isolate genes tagged by a high copy number transposable element. Plant Journal 7(4): 677-685.

Van den Broeck, D., Maes, T., Sauer, M., Zethof, J., Van Montagu, M. and Gerats, T. (1998). Transposon Display identifies individual transposable elements in high copy number lines. Plant Journal 13(1): 1 21-129.

Zwaal, R. R., Broecks, A., Van Meurs, J., Groenen, J. T. M., Plasterk, R. H. A. (1993). Target-selected gene inactivation in Caenorhabditis elegans by using a frozen transposon insertion mutant bank. PNAS 90(16): 7431-7435.

1. A method for parallel screening for gene insertion mutants simultaneously in a population of individuals of an organism, comprising:
preparing an insertion element mutant library representing the population of individuals, the insertion element mutant library comprising nucleic acid samples representing the population of individuals, wherein the insertion element mutant library is prepared from pools of individuals and includes a plurality of nucleic acid insertion elements and insertion element flanking sequences;
amplifying the insertion element flanking sequences with at least one primer derived from a sequence of a nucleic acid insertion element from the plurality of nucleic acid insertion elements, thus generating a set of amplified insertion element flanking sequences; and
hybridizing every sample of the set of amplified insertion element flanking sequences to a gene library comprising a gene, wherein the gene library is organized in at least a 2D array on a solid support, thus screening for the pools of individuals that contain insertions in the gene represented by the gene library.

2. The method according to claim 1, wherein preparing the insertion element mutant library comprises organizing the population in a 3D matrix, wherein the pools of individuals are formed by grouping the individuals in a fixed coordinate in one dimension of the 3D matrix and a varying coordinate in the other two dimensions of the 3D matrix.

3. The method according to claim 1, wherein amplifying the insertion element flanking sequences comprises iPCR using at least one primer or a set of primers based on a sequence of at least one nucleic acid insertion element of the plurality of nucleic acid insertion elements.

4. The method according to claim 3, wherein said iPCR comprises:
digesting the nucleic acid samples with at least one restriction enzyme resulting in a collection of amplifyable genomic fragments;
ligating at least one of the amplifyable genomic fragments by self ligation; and
amplifying the at least one amplifyable genomic fragment with a set of internal primers.

5. The method according to claim 4, further comprising reamplifying the at least one amplifyable genomic fragment with at least one primer based on a sequence of a nucleic acid insertion element of the plurality of nucleic acid insertion elements.

6. The method according to claim 1, wherein amplifying the insertion element flanking sequences comprises transposon display amplification.

7. The method according to claim 6, wherein the transposon display amplification comprises:
digesting the nucleic acid samples in the insertion element mutant library with a first restriction enzyme that recognizes six conserved nucleotides in the nucleic acid insertion element and a second restriction enzyme that recognizes a motif of four nucleotides, thus generating at least one restriction fragment including at least a tetracutter site, a hexacutter site, a part of an insertion element of the plurality of nucleic acid insertion elements, and at least part of the insertion element flanking sequence corresponding to the nucleic acid insertion element;
ligating a biotinylated adaptor to the hexacutter site of each of the at least one restriction fragment and a second adaptor to the tetracutter site of the at least one restriction fragments;
selecting biotinylated restriction fragments with magnetic streptavidin beads;
amplifying the insertion element flanking sequences with a composite primer comprising a 5′ part corresponding to the sequence of the biotinylated adaptor and a 3′ part corresponding to the sequence of the nucleic acid insertion element, including a part corresponding to the 5 last bases of the conserved hexacutter restriction site and extending several bases further into the sequence of the nucleic acid insertion element, in combination with a primer complementary to the second adaptor to perform a first round reaction; and
re-amplifying the insertion element flanking sequences with a nested primer based on the nucleic acid insertion element and a primer complementary to the second adaptor.

8. The method according to claim 1, wherein the solid support is a filter, micro-array, or chip.

9. The method according to claim 7, wherein the nucleic acid samples are selected from a group consisting of genomic DNA and cDNA.

10. The method according to claim 1, wherein the insertion element mutant library comprises 30 DNA samples from 100 organisms each.

11. The method according to claim 1, wherein the population is a cell line.

12. The method according to claim 1, further comprising labeling the set of amplified insertion element flanking sequences with a label.

13. The method according to claim 12, further comprising detecting any of the labeled amplification insertion element flanking sequences that hybridize to the gene library by visualizing a signal generated by the label.

14. A method for parallel simultaneous screening for one or more gene insertion mutants in a population of an organism, the method comprising:
preparing an insertion element mutant library comprising a plurality of nucleic acid insertion elements and insertion flanking sequences, wherein the insertion element flanking sequences originate from a defined population of an organism, and wherein the insertion element mutant library is built in at least a 2D array of pools;
amplifying the insertion element flanking sequences from said block, row and column pools with at least one primer derived from a sequence of a nucleic acid insertion element of the plurality of nucleic acid insertion elements; and
hybridizing the insertion element mutant library against a gene library organized in at least a 2D array of pools, wherein the hybridization is carried out pool of the insertion element mutant library against pool of the gene library.

15. The method according to claim 14, wherein the insertion element mutant library is built in 3D array of pools.

16. The method according to claim 14, wherein amplifying the insertion element flanking sequences comprises transposon display amplification.

17. The method according to claim 14, wherein the insertion element mutant library comprises 30 DNA samples from 100 organisms each.

18. The method according to claim 14, wherein the population is a cell line.

19. A method for parallel simultaneous screening for one or more gene insertion mutants in a population of an organism, the method comprising:
preparing an insertion element mutant library comprising a plurality of nucleic acid insertion elements and insertion flanking sequences, wherein the insertion element flanking sequences originate from a defined population of an organism, and wherein the insertion element mutant library is built in at least a 2D array of pools;
amplifying the insertion element flanking sequences from said block, row and column pools with at least one primer derived from a sequence of a nucleic acid insertion element of the plurality of nucleic acid insertion elements;
labeling the amplified insertion element flanking sequences with a label;
hybridizing the labeled, amplified insertion element flanking sequences against a gene library organized in at least a 2D array of pools, wherein the hybridization is carried out pool of the insertion element mutant library against pool of the gene library; and
detecting a gene insertion mutant by visualizing a signal generated by the label.
